(19) United States
(12) Patent Application Publication
(10) Pub. No.: US 2007/0265201 A1
(43) Pub. Date: Nov. 15, 2007

(54) INHIBITORS OF INFLAMMATORY CYTOKINE TRANSCRIPTION DERIVED FROM HCMV PROTEIN IE2

(76) Inventors: Philip E. Auron, Wexford, PA (US); James A. Listman, Manlius, NY (US)

Correspondence Address: BAKER BOTTS L.L.P., 30 ROCKEFELLER PLAZA, 44TH FLOOR, NEW YORK, NY, 10112-4498, US

(21) Appl. No.: 11/537,534

(22) Filed: Sep. 29, 2006

Related U.S. Application Data
(60) Provisional application No. 60/721,769, filed on Sep. 29, 2005.

(51) Int. Cl. A61K 38/16; A61K 38/04; A61P 1/00; A61P 11/08; A61P 17/00; A61P 19/02; A61P 27/06; A61P 29/00; A61P 3/04; A61P 31/12; A61P 35/00; A61P 37/06; A61P 37/08; A61P 7/04; A61P 9/10; C07K 14/045; C07K 7/00; C12N 5/02

(52) U.S. Cl. 514/12; 435/377; 514/13; 514/14; 530/324; 530/325; 530/326; 530/327

(57) ABSTRACT

The present invention provides novel inhibitors of inflammation and methods for treating inflammation by using these inhibitors. More specifically, the present invention provides transcription inhibitors, i.e., peptide fragments derived from internal regions of the Cytomegalovirus (CMV) IE2 protein that can inhibit production of inflammatory cytokines, e.g., Interleukin 1 beta(IFN-1β) and the methods for treating inflammatory diseases and CMV infection and related disorders, by using the IE2 fragments. An pharmaceutical composition comprising the transcription inhibitor are also provided.

### RELATED APPLICATION

The present application claims priority to U.S. Provisional Application Ser. No. 60/721,769, filed on Sep. 29, 2005, the contents of which are expressly incorporated herein.

### FIELD OF THE INVENTION

The present invention relates, at least in part, to novel inhibitors of inflammation and methods for treating inflammation using these inhibitors. More specifically, the present invention relates to peptide fragments derived from the Cytomegalovirus (CMV) IE2 protein, DNA encoding these peptides, peptidomimetics, and small molecules that can inhibit production of the inflammatory cytokine Interleukin 1 beta (IL-1β) and other inflammatory cytokines, and methods for treating inflammatory diseases by administering the peptide fragments.

### BACKGROUND OF THE INVENTION

Inflammation is a complex process that is characterized by a series of histological events and mediated by different forms of tissue injury, including both physical and infectious injuries. A subject body's defenses and repair mechanisms depend on IL-1β for function. While the inflammatory events are important for healing, prolonged or uncontrolled inflammation can lead to tissue injury and symptoms that cause illness. For example, septic shock, a leading cause of morbidity and mortality in hospitalized patients, results in part, by the action by IL-1β on vascular smooth muscle and myocardial function. Rheumatoid arthritis is a devastating chronic inflammatory disease and is characterized by the lesions being primarily confined to articular joints. Rheumatoid arthritis is a chronic systemic disease of the joints, marked by inflammatory changes in the synovial tissue and articular structures, and by atrophy and rarefaction of the bones. Cartilage and bone erosion are mediated by IL-1β (Dinarello, C. A. (2002) Clin Exp Rheumatol 20(5 Suppl 27): S1-13; van't Hof, R. J., K. J. Armour, et al. (2000) Proc Natl Acad Sci USA 97(14): 7993-8). Other conditions including atherosclerosis and ischemic reperfusion injury are also dependent on the action of IL-1β (Kato, A., C. Gabay, et al. (2002) Am J Pathol 161(5): 1797-803; Burne, M. J., A. Elghandour, et al. (2001) J Leukoc Biol 70(2): 192-8.; Haq, M., J. Norman, et al. (1998). J Am Soc Nephrol 9(4): 614-9); van't Hof, R. J., K. J. Armour, et al. (2000) Proc Natl Acad Sci USA 97(14): 7993-8).

IL-1β is a protein produced primarily by monocytes (though injured epithelial and endothelial cells also release small amounts), which are white blood cells that provide early warning signals to the body following injury. Various receptors on monocytes, such as Toll-like receptors, send signals to the nucleus that induce IL-1β production following stimulation. The IL-1β is then released into the surrounding environment and acts on both local and distant targets to alert the body's defenses. A well-recognized consequence of this stress signal is fever. In certain settings, such as septic shock and autoimmune diseases, including rheumatoid arthritis and Lupus, IL-1β contributes to the inflammation that causes severe injury to joints, bones, and other tissues. Blockade of the IL-1β protein's downstream function in rheumatoid arthritis has been shown to be of benefit. In animal models of solid organ transplantation, blockade of IL-1β function was shown to protect the transplant from the consequences of rejection. Other clinical trials have demonstrated a benefit to dental health by blockage of IL-1β, because chronic gingivitis leads to bone erosion and loss of dental stability under the influence of IL-1β. Therefore, there are a number of potential therapeutic uses for inhibitors of IL-1β

The strength of targeting IL-1β is that it disrupts inflammation at a very early stage. In fact, some of the pro-inflammatory affects of IL-1β are mediated by induction of cyclooxygenase (COX), the group of enzymes inhibited by NSAIDs, such as Viox™ and Celebrex™, to name a few. One advantage of the inhibitors of IL-1β of the present invention is that, unlike some COX inhibitors, IL-1β does not inhibit COX function. Therefore, there is no a priori reason to expect certain adverse events like myocardial infarction as a result of inhibiting IL-1β expression.

The IL-1β promoter is a region of DNA that is located in front of the IL-1β gene and regulates transcription by binding a series of proteins called transcription factors. Transcription factors contain DNA binding domains that bind specific DNA sequences present in the promoter. When the binding occurs, other proteins necessary for transcription, called transactivators, are recruited to the promoter by binding to other domains on the surfaces of the transcription factors. In monocytes, a key transcription factor for the IL-1β promoter is Spi-1 (also referred to as PU.1). Spi-1 is a member of the ETS family of transcription factors and plays a pivotal role in lineage commitment during hematopoesis and is important for transactivation of multiple effector genes within the immune system including TNFα, IL-6, and myeloperoxidase, among others (Friedman, A. D. (2002) Oncogene 21(21): 3377-90). Spi-1 binds to two separate target sites on the IL-1β promoter and may also require another transcription factor, C/EBPβ, to function (Kominato, Y., D. Galson, et al. (1995) Mol Cell Biol 15(1): 59-68).

It has been found that the Cytomegalovirus (CMV) infection of monocytic cell lines results in induction of IL-1β (Iwamoto, G. K., M. M. Monick, et al. (1990). J Clin Invest 85(6): 1853-7). This observation was later explained by the ability of the CMV encoded transactivator Immediate Early 2 protein (IE2), to induce transcription of the IL-1β gene when co-transfected into cells with Spi-1. Furthermore, IE2 activity was explained by its ability to physically interact with Spi-1 on the IL-1 promoter. (Wara-aswapati, N., Z. Yang, et al. (1999). Mol Cell Biol 19(10): 6803-14).

Numerous anti-inflammatory treatments are known and commonly used. The most common are aspirin and the nonsteroidal anti-inflammatory agents such as naproxen, ibuprofin, diflunisal, mefenamic acid, and ketorolac tromethamine. These agents generally are used to treat short-term mild inflammation and pain. More severe inflammatory diseases, such as arthritis, are treated with glucocorticoids and antagonistic antibodies. The former are generally not well tolerated chronically due to many side effects and the latter are very expensive to produce.

Anti-tumor necrosis factor (TNF) monoclonal antibodies are available which reduce inflammation and are currently marketed for the treatment of rheumatoid arthritis such as Remicade™ and Humira™. However, it has been recently found that administration of these antibodies may increase the risk of infection as well as cancer (Bongartz et al. (2006) J. of the American Medical Association 295;2275).

Currently, there are only a few specific inhibitors of IL-1β available. Unfortunately, these inhibitors only block IL-1β by binding to its receptors on target cells after IL-1β has already been synthesized, released and begins to function. This type of IL-1β inhibition may be too late in some circumstances of inflammation or may minimize the effectiveness of treatment. Since many of the anti-inflammatory agents are only short acting, often produce severe side effects (e.g., glucocorticoids) and/or are expensive to produce (e.g., monoclonal antibodies), a need for new therapies has arisen.

### SUMMARY OF THE INVENTION

The present invention is based, at least in part, on the discovery that peptide fragments derived from an internal region of the Cytomegalovirus (CMV) immediate early 2 (IE2) protein inhibit production of the inflammatory cytokine Interleukin 1 beta (IL-1β), and block or inhibit the function of intact IE2. The present invention provides a novel approach to blocking, and thereby inhibiting, preventing and treating inflammation by using competitive antagonists that interfere with expression of molecules whose expression is modulated by Spi-1, such as proinflammatory cytokines, e.g., IL-1β and tumor necrosis factor (TNF).

Cytomegalovirus (CMV) infection of monocytic cell lines results in induction of IL-1β (Iwamoto, G. K., M. M. Monick, et al. (1990) J Clin Invest 85(6): 1853-7). This induction results from the ability of the CMV encoded transactivator, Immediate Early 2 protein (IE2), to induce transcription of the IL-1β gene upon binding to Spi-1 (Wara-aswapati, N., Z. Yang, et al. (1999) Mol Cell Biol 19(10): 6803-14).

The IE2 protein aids in CMV replication in cells. An example of an amino acid sequence of CMV IE2 is set forth as SEQ ID NO:4 and an example of the nucleotide sequence of CMV IE2 is set forth as SEQ ID NO:5 (see also Stenberg, R M, Depto, A S, et al (1989) J Virol 63(6): 2699-2708; Stenberg R M, Witte, P R, et al (1985) J Virol 56(3): 665-675; and Chee, M. S., Bankier, A. T, et al (1990) Curr. Top. Microbiol. Immunol. 154: 125-169). IE2 is a potent viral and host cell gene transactivator that has both DNA and protein binding activities. IE2 induces transcription of the IL-1β gene when co-transfected into cells with Spi-1. The transcription factor C/EBPβ is also involved in transcription of the IL-1β gene.

The function of Spi-1 and C/EBPβ on the IL-1β promoter is demonstrated by gene reporter assays (FIGS. 1B and 1C). In these studies, HeLa S3 cells (a non myelocytic cell line that does not express Spi-1) were transiently transfected with a reporter vector containing the IL-1β promoter linked to the firefly luciferase gene. This vector expresses the luciferase reporter in a titratable fashion when eukaryotic expression vectors containing cDNA for these transcription factors are added. Superactivation of the IL-1β promoter by IE2 has been observed when both Spi-1 and C/EBPβ are coexpressed in a reporter assay (FIG. 1D).

The present invention is based, at least in part, on the surprising discovery that peptide fragments of the CMV IE2 protein can function as competitive antagonists to block or inhibit the function of native transcription factors required for transcription of proinflammatory cytokines, such as IL-1β, TNF (e.g., TNFα,) IL-6, and also myeloperoxidase, and other molecules whose transcription is modulated by Spi-1, to thereby inhibit expression of these cytokines and other molecules, and also to block or inhibit the function of intact IE2.

By inhibiting the early stage of inflammation, the present invention offers an advantage over commercially available anti-inflammatory agents or antagonists, such as glucocorticoids or monoclonal antibodies, which cannot stop the earliest events in the inflammation cascade and which also exhibit significant side effects when used chronically or are expensive to produce.

Accordingly, one aspect of the invention is directed to isolated polypeptide fragments of CMV IE2 protein (e.g., IE2 291-364 and IE2 291-343 of SEQ ID NO:4), which have the ability to inhibit inflammation by blocking expression of IL-1β and other proinflammatory cytokines. Another aspect of the invention is directed to a smaller peptide that comprises a minimal Spi-1 interaction region (IE2 315-328 of SEQ ID NO:4) contained within the IE2 291-364 polypeptide. Thus, in one aspect, the present invention is directed to an isolated polypeptide that comprises Cytomegalovirus (CMV) immediate early 2 (IE2) protein amino acid residues 315-328 (IE2 315-328 of SEQ ID NO:4), preferably, human CMV (hCMV) IE2 315-328 of SEQ ID NO:4. In a preferred aspect, the polypeptide of the present invention is an IE2 protein fragment that comprises or consists of hCMV IE2 protein amino acid residues 291-364 (IE2 291-364), hCMV IE2 protein amino acid residues 291-343 (IE2 291-343) and hCMV IE2 protein amino acid residues 315-328 (IE2 315-328). The present invention also provides polypeptides that are fragments of the above-mentioned polypeptides, and polypeptides that are at least about 80%, 81%, 82%, 83%, 84%, 85%, 86%, 87%, 88%, 89%, 90%, 91%, 92%, 93%, 94%, 95%, 96%, 97%, 98%, 99%, or more homologous to the above-mentioned peptides, as well as derivates thereof, wherein the polypeptide retains the ability to bind Spi-1 and/or inhibit transcription of an Spi-1-modulated molecule, such as a proinflammatory cytokine, e.g., IL-1β or TNF.

In another aspect, the present invention is directed to an E2 polypeptide/IE2 fragment that comprises hCMV IE2 protein amino acid residues 315-328 (IE2 315-328), and is at least about 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 150, 160, 170, 180, 190, 200, or more amino acids in length, up to less than the full length amino acid sequence of E2, and retains the ability to bind Spi-1 and/or inhibit transcription of an Spi-1-modulated molecule, such as a proinflammatory cytokine, e.g., IL-1β or TNF.

The present invention is also directed to nucleic acid molecules that encode the polypeptide/IE2 fragments of the invention.

The present invention is not limited to E2 peptide fragments. Transcription inhibitors of the invention also include peptidomimetics and small molecules that function to bind Spi-1 and/or inhibit transcription of a Spi-1-modulated molecule Spi-1-modulated molecule.

Still another aspect of the present invention is directed to a pharmaceutical composition containing a therapeutically effective amount of a transcription inhibitor of the present invention and a pharmaceutically acceptable carrier.

In one aspect, the present invention provides a method of inhibiting inflammation by administering to a subject in need thereof a sufficient amount of a transcription inhibitor or a pharmaceutical composition of the present invention.

In another aspect, the present invention provides a method for treating an inflammatory disorder by administering to a subject in need thereof a therapeutically effective amount of a transcription inhibitor or a pharmaceutical composition of the present invention. The inflammatory disorders contemplated by the present invention include, but are not limited to, arthritis, rheumatoid arthritis, autoimmune diseases (e.g., inflammatory bowel disease and Systemic Lupus Erythematosus), solid organ transplantation, acute infection, acute phase response, allergic asthma, anorexia, asthma, cachexia, cardiovascular effects, coagulation, fever, gingivitis, graft versus host disease, hemorrhage, multiple sclerosis, neovascular glaucoma, osteoarthritis, periodontitis, psoriasis, psoriatic arthritis, rheumatic fever, shock, and solid tumor growth and tumor invasion by secondary metastases.

In yet another aspect, the present invention provides a method for treating inflammatory disorders by administering to a subject in need thereof a therapeutically effective amount of a transcription inhibitor or a pharmaceutical composition of the present invention in combination with one or more additional anti-inflammatory agents including, but not limited to, selective COX-2 inhibitors, interleukin-1 antagonists, dihydroorotate synthase inhibitors, p38 MAP kinase inhibitors, TNF-α inhibitors, TNF-α sequestration agents, and methotrexate. This approach can yield success in diseases like septic shock where treatments with individual inhibitors have not demonstrated significant improvement in outcomes in human trials (Vincent, J. L. (1997). “New therapies in sepsis.” Chest 112(6): 330S-338).

In still another aspect, the present invention provides methods for inhibiting expression of a proinflammatory cytokine, e.g., IL-1β or TNF in a cell comprising contacting the cell, e.g., a monocyte, with a sufficient amount of a transcription inhibitor, e.g., a polypeptide/IE2 fragment.

In yet another aspect, the invention provides methods for treating a disease or disorder associated with CMV infection in a subject comprising administering to the subject a therapeutically effective amount of a transcription inhibitor, e.g., a polypeptide/IE2 fragment or a pharmaceutical composition of the present invention. In one embodiment, the disease or disorder associated with CMV is for example: CMV retinitis, hepatitis, CMV-associated acute transverse myelitis, mononeuropathy multiplex, encephalitis, fever, rash, or fatigue.

In another aspect, the present invention provides methods for inhibiting CMV expression in a cell comprising contacting the cell with a transcription inhibitor, e.g., a polypeptide/IE2 fragment of the invention.

In still another embodiment, the present invention provides methods for treating, preventing, or limiting a CMV viral infection in a subject comprising administering to the subject a therapeutically effective amount of a transcription inhibitor, e.g., a polypeptide/IE2 fragment, a pharmaceutical composition of the present invention.

### BRIEF DESCRIPTION OF THE DRAWINGS

FIGS. 1A-1D depict cooperative transactivation of the IL-1B promoter by Spi-1, C/EBPβ and IE2. FIG. 1A is a schematic of the IL-1B promoter showing two composite Spi-1-C/EBPβ binding sites in proximity to the transcription start site. The full-length promoter (HT) was spliced into a vector containing the gene reporter firefly luciferase and used in transient transfection assays in HeLa-S3 cells. FIG. 1B depicts dose-response of HT reporter to titrated Spi-1 expression vector and a fixed amount of cotransfected C/EBPβ expression vector. FIG. 1C depicts dose-response of HT reporter to titrated C/EBPβ expression vector and a fixed amount of Spi-1 expression vector. FIG. 1D depicts functional cooperativity with IE2. The HT reporter was contransfected with titrated Spi-1 expression vector (open bars) or fixed amount of C/EBPβ expression vector (checkerboard bars), or both C/EBPβ and IE2 expression vectors (hatched bars).

FIGS. 2A-2B show that the cooperative transactivation of the IL-1β promoter is inhibited by the IE2 derived fragments IE2 291-364 and IE2 291-343. The data shown here are pooled from three independent experiments. FIG. 2A shows that the HT reporter (as described in FIG. 1) was transiently transfected with Spi-1 and C/EBP expression vectors into Hela S3 cells. The indicated transfection groups were con-transfected with titrated amounts of IE2 291-364 or 291-343 expression vectors. FIG. 2B shows the inhibitory effects of the IE2 fragments on exogenous regulation of the IL-1β promoter by wild type IE2. The experiment described by FIG. 2B is similar to that described by FIG. 2A except that wild type IE2 was also added to the indicated transfection groups. These are pooled data from three independent experiments.

FIG. 3 depicts that IE2 291-364 fragment inhibits full-length IL-1β reporter in the monocytic cell line RAW264.7. A luciferase gene reporter was made by splicing the native IL-1β upstream fragment (XT), which contains the enhancer through promoter sequences, upstream of the luciferase gene. This was cotransfected (in titrated doses) with pCDNA 3.1/V5-His empty vector (control) or pCDNA 3.1/V5-His IE2 291-364 by calcium phosphate precipitation into RAW 264.7 cells. To activate the reporter, cells were stimulated with LPS.

FIGS. 4A-4D. FIG. 4A depicts both cytoplasmic and nuclear localization of a GFP-IE2 291-343 fusion protein following transfection into HeLa-S3 cells. FIG. 4B depicts GFP IE2 291-343 inhibition of IL-1β promoter activity. FIG. 4C is a specificity control showing that the peptide E2 291-364 does not inhibit expression of C/EBPβ by western blot analysis of cell lystates from HeLa-S3 transfected as described in FIG. 2B. FIG. 4D is another specificity control showing that a control GFP expression vector was not affected by either IE2 291-343 or IE2 291-364. HeLa-S3 cells were transfected with both a GFP expression vector and cotransfected with either E2 291-343 or IE 291-364 expression vectors and GFP expression analyzed 24 hours later by a fluorescence activated cell sorter.

FIG. 5 demonstrates loss of interaction of IE2, Spi-1 ETS domain, and C/EBPβ bZIP domain radiolabeled probes to an IE2 291-364 fragment with an internal deletion of residues 315-328 (delA). Interaction of the indicated probes is retained to the control peptides IE2 291-343 and IE2 291-364. This result indicates that the IE2 region 315-328 confers binding to these ligands and might be a minimal peptide inhibitor.

FIG. 6 depicts increasing amounts of vector expressing either IE2 291-364Δ315-328 or IE2 291-364 transfected into RAW cells with full length IL-1β regulatory protein as a luciferase reporter. After transient transfection by CaPO4, RAW cell lysates were used for measurement by luminometry and results normalized to β-galactosidase activity introduced by a co-transfected β-galactosidase expression vector to control for transfection efficiency. Values for IE2 291-364Δ315-328 are reported as percentages of relative luciferase activity compared to the wild-type fragment 315-264, as shown.

FIG. 7 depicts an example of an amino acid and nucleotide sequences of the IE2 molecule (SEQ ID NO:4) and an example of a nuleotide sequence encoding the fragments of the invention (SEQ ID NO:5). FIG. 7 also contains examples of IE2 peptide fragments as set forth as SEQ ID NOs:1, 2, and 3. The amino acid sequence is contained in GenBank Accession No. P19893 (GI:59803018) and the nucleotide sequence is contained within GenBank Accession No. Ml 1298 (GI:330552). The present invention is not limited to E2 sequences derived from any particular strain of CMV. Additional variations of the amino acid and nucleotide sequences are publicly available.

### DETAILED DESCRIPTION OF THE INVENTION

The present invention is based, at least in part, on the discovery that peptide fragments derived from an internal region of the Cytomegalovirus (CMV) immediate early 2 (IE2) protein (for example, human CMV (hCMV) IE2 fragments comprising IE2 amino acids 315-328 of SEQ ID NO:4, e.g., IE2 amino acids 291-364 or 291-343 of SEQ ID NO:4), inhibit production of the inflammatory cytokine Interleukin 1 beta (IL-1β), and block or inhibit the function of intact IE2. Since IL-1β is involved in one the earliest steps in the inflammation cascade, the inhibition of IL-1β achieved by the present invention in turn can prevent and treat inflammation or inflammatory diseases.

Since key proinflammatory genes in addition to IL-1β (including that coding for tumor necrosis factor (TNF)), require transcription factors similar to Spi-1, in a particular embodiment, the present invention contemplates that transcription inhibitors of the present invention can also block expression of these additional molecules, including, but not limited to, TNF, e.g., TNFα, IL-6, and the lysosomal enzyme myeloperoxidase. Thus, the transcription inhibitors of the present invention are advantageous anti-inflammatory agents, since many of the other anti-inflammatory agents block only one pathway in inflammation.

According to one embodiment of the present invention, transcription inhibitors of the present invention are small antagonistic peptides that inhibit IL-1β production at the level of gene transcription, the initial process of converting the genetic code into a protein. Without intending to be limited by a particular theory, it is believed that by reducing IL-1β production at an early stage of the inflammatory cascade, there will be a better chance to inhibit certain types of inflammatory responses for therapeutic benefit. It is also believed that smaller peptides, such as IE2 fragments, are easier, and thus, cheaper to produce, thus making the present invention an attractive choice for pharmaceutical development. It is also contemplated that an IE2 fragment provided by the present invention and its identified target transcription factor interaction sites can be used as a basis for developing even smaller molecules that could serve as an additional class of novel anti-inflammatory drugs.

Also without intending to be limited by a particular theory, it is believed that an IE2 fragment of the present invention binds to Spi-1, but does not function to activate transcription of a gene because the IE2 fragment is missing the functional components of IE2 protein that are necessary for transcription to occur. It is believed that the IE2 fragments compete with intact functional IE2 proteins for binding to Spi-1 and thereby inhibit transcription of inflammatory cytokines, e.g., IL-1β and other Spi-1-modulated molecules. It is also believed that the IE2 peptide fragments may be effective as anti-viral agents by inhibiting CMV expression within infected cells, since it was demonstrated that the IE2 peptide can also block IE2-dependent gene action.

As used herein, the term “transcription inhibitor” or “antagonist” is meant to include any molecule that is capable of inhibiting transcription of an Spi-1-modulated gene, such as, but not limited to IL-1β, TNF, e.g., TNFα, IL-6, and myeloperoxidase. An antagonist can include, for example, an IE2 protein fragment, a peptidomimetic, a nucleic acid molecule, or small molecule of the invention. A transcription inhibitor can also be used to inhibit CMV expression and/or infection. As used herein, the term “anti-inflammatory agent” of the invention includes any of the above antagonists and is not limited to a peptide.

By “inflammatory disorder,” “inflammatory disease” or “inflammatory condition” is meant a condition accompanied by or tending to cause a local response to cellular injury that is marked by capillary dilatation, leukocytic infiltration, redness, heat, pain, swelling, and often loss of function. A mammal body's defenses and repair mechanisms in an “inflammatory disorder,” “inflammatory disease” or “inflammatory condition” are believed to depend on IL-1β for function. An “inflammatory disorder,” “inflammatory disease” or “inflammatory condition” also includes an autoimmune disease or disorder.

By “a disease or disorder associated with CMV infection” is meant any disease, disorder, or condition related to or caused by infection by CMV. Diseases or disorders associated with CMV infection include, but are not limited to, CMV retinitis, hepatitis, CMV-associated acute transverse myelitis, mononeuropathy multiplex, encephalitis, fever, rash, and fatigue.

By “IE2 fragment” is meant a peptide fragment that is derived from any Immediate Early 2 (IE2) protein and does not induce transcription of a proinflammatory cytokine. In IE2 fragment is preferably a peptide fragment comprising human Cytomegalovirus (hCMV) IE2 protein amino acid residues 315-328 but not including any functional components in the IE2 protein that are necessary for induction of transcription of pro-inflammatory genes (e.g., IL-1β and TNF). More preferably, a peptide fragment derived from the hCMV IE2 protein region ranging from amino acid residue 291 to amino acid residue 364 of SEQ ID NO:4 and comprising amino acid residues 315-328 of SEQ ID NO:4 is contemplated by the present invention.

By “therapeutically effective amount” is meant the dose required to treat a condition or disease, particularly, an inflammatory condition or disease. “Therapeutically effective amount” is intended to include an amount of a polypeptide/IE2 fragment of the present invention or an amount of the combination of polypeptide/IE2 fragments claimed effective to inhibit inflammation or CMV infection in a cell.

The term “treatment” or “treat” is meant the treatment of a disease-state, e.g., an inflammatory disease or disorder or infection, in a mammal, particularly in a human, and include: (a) preventing the disease-state from occurring in the mammal, in particular, when the mammal is predisposed to the disease-state but has not yet been diagnosed as having the disease or condition; (b) inhibiting the disease-state, i.e., arresting its development; (c) decreasing, relieving or ameliorating the disease-state or infection, or the clinical symptoms thereof, i.e., causing regression of the disease state; and/or (d) shortening the duration of the disease.

To “prevent” a disease-state, as that term is used herein, means that, when administered to an individual who has not yet been diagnosed as suffering from a disease-state, e.g., an inflammatory disease or infection, will decrease the risk that that individual will develop a diagnosis of the disease-state.

To “limit” infection, as that term is used herein, means to limit the spread of infection within an individual and/or between individuals.

By “subject” is referred to any mammalian subject, preferably, a human.

I. Polypeptides

According to the present invention, a peptide fragment of the IE2 protein, for example, between amino acid regions 291-364 of SEQ ID NO:4, can inhibit Spi-1 function on the IL-1β promoter. In one embodiment, a peptide fragment of the invention that interacts with Spi-1 is the 14 amino acid region (IE2 315-328) within amino acids 291-264 of IE2.

Another embodiment of the present invention is directed to an anti-inflammatory agent that comprises at least one polypeptide/IE2 fragment of the present invention.

Without intending to be limited by a particular theory, it is believed that the IE2 fragment contains the Spi-1 ETS domain (DNA binding domain) interaction region and retain ternary structural integrity in the presence of a glutathione-S-transferase (GST) or green fluorescence protein (GFP) backbone. Because the IE2 fragments lack most of the transactivation domain of E2, it is believed that the IE2 fragments confer a dominant negative function by blocking cooperative IE2/Spi-1 interaction on the IL-1β promoter to thereby reduce transcription of IL-1β and thus function as anti-inflammatory agents.

One embodiment of the present invention is directed to an isolated or purified polypeptide that comprises Cytomegalovirus (CMV) immediate early 2 (IE2) protein amino acid residues 315-328 (IE2 315-328) (SEQ ID NO: 1), preferably, human CMV (hCMV) IE2 315-328, wherein the polypeptide inhibits inflammation. In a preferred embodiment, the polypeptide of the present invention is a IE2 protein fragment that includes, but is not limited to, hCMV IE2 protein amino acid residues 291-364 (IE2 291-364) (SEQ ID NO: 2). A polypeptide of the present invention also includes any IE2 protein fragment contained within amino acids 291-364 of IE2 that retains the ability to bind to Spi-1 and/or inhibit transcription of an Spi-1-modulated molecule, e.g., IL-1β or TNF, such as, for example, hCMV IE2 protein amino acid residues 291-343 (IE2 291-343) (SEQ ID NO: 3) and hCMV IE2 protein amino acid residues 315-328 (IE2 315-328) (SEQ ID NO: 1). In addition, the polypeptide fragment of the invention can be at least about 14, 15, 16, 17, 18, 19, 20, 25, 30, 35, 40, 45, 50, 55, 60, 65, 70, 75, or more amino acids, but less than the full length IE2 protein. The fragment may also comprise additional amino acid residues outside of amino acids 291-364 of IE2 that do not affect the inhibitory function of the polypeptide.

A polypeptide of the present invention also includes derivatives or analogs of the above-mentioned polypeptides, in which one or more of the amino acids in an above-listed sequences are substituted with another natural or unnatural amino acid, wherein the polypeptide retains its activity, e.g., the ability to bind to Spi-1 and/or inhibit transcription of an Spi-1-modulated molecule, e.g., IL-1β or TNF. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutarine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In certain embodiments homologous substitution may occur, which is a substitution or replacement of like amino acids, such as basic for basic, acidic for acidic, polar for polar amino acids, for example. Non-homologous substitution may also occur i.e., from one class of residue to another or alternatively involving the inclusion of unnatural amino acids such as ornithine, diaminobutyric acid ornithine, norleucine ornithine, pyriylalanine, thienylalanine, naphthylalanine and phenylglycine. Amino acid substitutions can be selected to enhance the hydrophobicity of the variant peptide, the amphipathic nature of a variant peptide, and to enhance or decrease the probability that a variant peptide forms an alpha-helical structure or substructure.

In other embodiments, a polypeptide of the invention is substantially identical to the amino acid sequences set forth above, and retains the ability to bind to Spi-1 and/or inhibit transcription of an Spi-1-modulated molecule, e.g., a proinflammatory cytokine, such as, but not limited to IL-1β or TNF, yet differs in amino acid sequence due to natural allelic variation or mutagenesis. In another embodiment, a polypeptide of the invention comprises an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or more homologous to the above-mentioned peptides.

The polypeptide/IE2 fragment or the anti-inflammatory agent of the present invention may be prepared by known recombinant molecular biology procedures (e.g., Mullis et al., Methods Enzymol. 155: 335-50 (1987) and Ausubel et al., Current Protocols in Molecular Biology, for example pages 3.17.1-10). A polypeptide of the present invention also may be synthesized by peptide ligation methods (see, e.g., Dawson et al., Science 266: 776-9 (1994) and Coligan et al., Native chemical ligation of polypeptides, Wiley: 18.4.1-21 (2000)). Polypeptides, mimetics and variants thereof may be produced by standard chemical synthetic methods known in the art (e.g., peptide synthesizer commercially available from Applied Biosystems).

The polypeptides of the present invention may be isolated using standard purification procedures. An “isolated” or “purified” peptide, polypeptide or protein is substantially free of cellular material or other contaminating proteins from the cell or source from which the protein is derived, or substantially free from chemical precursors or other chemicals when chemically synthesized. “Substantially free” means preparation of a polypeptide or variant thereof having less than about 30%, 20%, 10% and more preferably 5% (by dry weight), of non-IE2 polypeptides (also referred to herein as a “contaminating protein”), or of chemical precursors or non-receptor or ligand chemicals. When the polypeptide or a biologically active portion thereof is produced recombinantly, it often is substantially free of culture medium, specifically, where culture medium represents less than about 20%, less than about 10%, and often less than about 5% of the volume of the polypeptide preparation. Isolated or purified polypeptide preparations may be 0.01 milligrams or more or 0.1 milligrams or more, and often 1.0 milligrams or more and 10 milligrams or more in dry weight.

Also included in the present invention are IE2 peptide fragments fused to an additional compound such as, for example, a protein transduction domain (PTD), peptides which are PEGylated (bearing one or more linked polyethylene glycol molecule), or nuclear localization signal (NLS). Furthermore, IE2 fragments of the invention may be linked to a second protein or comprised within a larger peptide where the larger peptide is not IE2, e.g., to increase stability in a cell.

In addition to the peptides described herein, the transcription inhibitors of the invention also include peptidomimetics, which are small, peptide-like molecules which mimic the inhibitory activity of the IE2 polypeptide fragments.

II. Nucleic Acid Molecules

Another aspect of the invention pertains to isolated nucleic acid molecules encoding the polypeptides of the invention. For example, the present invention includes fragments of the IE2 nucleic acid molecule encoding hCMV E2 protein amino acid residues 291-364 (IE2 291-364) (SEQ ID NO: 2), hCMV IE2 protein amino acid residues 291-343 (IE2 291-343) (SEQ ID NO: 3), and hCMV IE2 protein amino acid residues 315-328 (IE2 315-328) (SEQ ID NO: 1), and complements thereof. Fragments of these isolated nucleic acid molecules that encode polypeptides that retain the ability to bind to Spi-1 and/or inhibit transcription of an Spi-1-modulated molecule, e.g., IL-1β or TNF, are also included in the present invention, as are isolated nucleic acid molecules comprising a nucleotide sequence which is at least about 80%, 81%, 82%, 83%, 84%, 85%, 86%, 87%, 88%, 89%, 90%, 91%, 92%, 93%, 94%, 95%, 96%, 97%, 98%, 99%, or more identical to the entire length of the nucleotide sequences that encode the above-mentioned polypeptides, or a portion of any of these nucleotide sequences, that retains the ability to bind to Spi-1 and/or inhibit transcription of an Spi-1-modulated molecule, e.g., a proinflammatory cytokine, and natural allelic variants and homologues thereof.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the DNA which the nucleic acid is derived. Moreover, an “isolated” nucleic acid molecule can be substantially free of other cellular material, or culture medium when produced by recombinant techniques, or substantially free of chemical precursors or other chemicals when chemically synthesized.

III. Additional Anti-Inflammatory Agents

In a particular embodiment, the present invention also contemplates a method of drug design for inhibiting production of IL-1β, comprising co-crystallizing the IE2 315-328 and Spi-1 ETS domain using any crystallization set up well-established in the art including, but not limited to, hanging drop, sitting drop, free liquid diffusion, batch or micro-batch, obtaining a three-dimensional representation of at least one binding site of IE2 315-328 on the Spi-1 ETS domain protein, superimposing at least one candidate ligand compound, preferably, a ligand compound having a molecular size smaller than amino acids 315-328 of IE2, on the three dimensional representation of the ligand binding site, evaluating the binding between the candidate compound and the binding site, and selecting a compound that spatially fits the ligand binding site.

IV. Pharmaceutical Compositions and Administration Thereof

In another embodiment, the present invention is directed to a pharmaceutical composition containing a therapeutically effective amount of at least one transcription inhibitor of the invention, e.g., a polypeptide/IE2 fragment, a DNA molecule encoding a polypeptide/IE2 fragment, a peptidomimetic, or a small molecule of the present invention, or a combination thereof, and a pharmaceutically acceptable carrier.

The active ingredients of a pharmaceutical composition containing an IE2 fragment or a nucleic acid encoding the IE2 fragment or other transcription inhibitors of the invention transcription inhibitor are contemplated to exhibit effective therapeutic activity, for example, in inhibiting inflammation, and treating inflammatory disorders or diseases or CMV infection. Thus the active ingredients of the therapeutic compositions containing transcription inhibitors, e.g., IE2 fragments, are administered in therapeutic amounts which depend on the particular disease. As non-limiting, specific examples, an effective amount may be in the range of between about 1 mg and 1 g/kg, between about 1-100 mg/kg, or between about 5 and 20 mg/kg, depending upon the weight of the subject. For example, peak blood concentrations of IE2 fragments for treating rheumatoid arthritis can be about 5 to 100 mcg/ml using 100 mg to 1000 mg per day. The dosage regimen can be adjusted to provide the optimum therapeutic response. A pharmaceutical composition according to the invention may comprise a partial dose, a single dose, or multiple doses. For example, several divided doses can be administered daily or the dose can be proportionally reduced as indicated by the exigencies of the therapeutic situation. In some embodiments, the composition is administered locally, e.g., for the treatment of arthritis, or systemically.

Administration of one or more forms of the transcription inhibitors such as IE2 315-328, IE2 291-343, and IE2 291-364, are contemplated. In one embodiment, the IE2 polypeptide fragments of the invention are delivered to a cell by linking them to an additional transduction sequence, e.g., a protein transduction domain (PTD), to facilitate transport of the peptide across cell membranes. A protein transduction domain is a small protein domain that cross biological membranes efficiently and independently of transporters or specific receptors, and promote the delivery of peptides and proteins, DNA, and other compounds into cells. Examples of PTDs are, for example, domains of the TAT protein from human immunodeficiency virus (HIV-1), the third alpha-helix of Antennapedia homeodomain, VP22 protein from herpes simplex virus, and poly-arginine domains. For example, the published Tat transduction signal peptide sequence YARAAAAQARA (SEQ ID NO:6) may be utilized. Other PTDs are known in the art and are also described in, for example, Mi, Z. et al. (2000) Mol. Therapy (4):339-47; Ryu, J et al. (2003) Mol Cells., 16(3):385-91; Matsui, et al. (2003) Curr Protein Pept Sci. (2):151; Matsui et al. (2003) Nippon Yakurigaku Zasshi 121(6):435; Dietz, G. P. and Bahr, M. (2004) Mol. Cell. Neurosci. 27(2):85; Torchilin (2006) Annual Review of Biomedical Engineering 8: 343-375; and Harada et al. (2006) Breast Cancer 13(1):16. Additional PTDs are described in U.S. Pat. No. 6,881,825, and U.S. Patent Application Serial Nos. 20030104622A1, 20030219826A1, and 20050074884A1. Another peptide carrier, called Pep-1, has also been described and may be used to deliver the polypeptides of the invention. Morris, M. et al. Nat Biotechnol. 2001 (12):1173-6. The contents of all the above are expressly incorporated herein by reference.

Depending on the route of administration, the active ingredients, which comprise the transcription inhibitors, may be required to be coated in a material to protect the ingredients from the action of acids and other natural conditions which may inactivate the ingredients. For example, the transcription inhibitors c an be administered in an adjuvant or in liposomes, microspheres, nanoparticles (see Shinji et al., 1997, Int. J. Pharmacol. 149:93-106) or microcapsule, for example, to protect it from degradation by proteases (see, e.g., Arhewoh et al., 2005, African J. Biotechnol 4:1591-1597). Adjuvants contemplated herein include resorcinols, non-ionic surfactants such as polyoxyethylene oleyl ether and n-hexadecyl polyethylene ether. Liposomes include water-in-oil-in-water P40 emulsions as well as conventional liposomes. Further specific non-limiting examples include encapsulation of a formulation comprising the transcription inhibitor in a chitosan-coated alginate bead, the transcription inhibitor in a hydrogel formulation (see, e.g., Blanchette et al., 2004, Biomed. & Pharmacother. 58:142-151), the transcription inhibitor comprised in a liquid or a dry powder formulation adapted for pulmonary administration (see, for example, Patton, 1997, Chemtech 27(12)27(12):34-38 and Patton, 1998, Nature Biotechnol. 16:141-143), the transcription inhibitor incorporated in a matrix release device (see Krishnaiah et al., 2001, J. Controlled Rel. 77:87-95) or in pro-drug form (see Yano et al., 2002, J. Controlled Rel. 79:103-112.

Under ordinary conditions of storage and use, the preparations of the present invention contain a preservative to prevent the growth of microorganisms. The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients (i.e., IE2 fragments or DNA molecules encoding the IE2 fragments or a combination thereof) into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yield a powder of the active ingredient plus any additional desired ingredient from previously sterile-filtered solution thereof.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of the active material calculated to produce the desired therapeutic effect in association with the required pharmaceutical carrier. The specification for the novel dosage unit forms of the invention are dictated by and directly depending on (a) the unique characteristics of the active material and the particular therapeutic effect to be achieved, and (b) the limitations inherent in the art of compounding such as active material for the treatment of injury in living subjects having a condition in which bodily health is impaired as herein disclosed in detail.

The principal active ingredient is compounded for convenient and effective administration in effective amounts with a suitable pharmaceutically acceptable carrier in dosage unit form as hereinbefore disclosed. A unit dosage form can, for example, result in achieving blood concentrations of IE2 peptide fragments of the invention, for example, of about 2 mg to about 30 mg/ml at a dosage of about 100 mg to about 1000 mg per day.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, intraarticular, intrathecal, introcular, intraventricular, and rectal administration. As used herein “pharmaceutically acceptable carrier” includes any and all solvents, dispersion media, coatings, antibacterial and antifungal agents, isotonic and adsorption delaying agents, and the like. The use of such media agents for pharmaceutically active substances is well known in the art. Except insofar as any conventional media or agent is incompatible with the active ingredient, use thereof in the therapeutic compositions is contemplated. Supplementary active ingredients can also be incorporated into the compositions.

A pharmaceutical composition, as set forth, may be contained within a medical device. In one non-limiting example, the device is a syringe. In another non-limiting example, the device is an inhaler, where, in specific examples, the inhaler is capable of delivering a positive pressure (see U.S. Pat. No. 6,708,688).

Administration of a transcription inhibitor, e.g., a IE2 fragment, can also include altered forms or derivatives of the transcription inhibitor or drugs that enhance its activity, stability, or accessibility in a subject. The identification of applicable transcription inhibitor enhancing drugs are readily tested or screened by method for drug design using co-crystallization as described above or by examining the effects of the drugs or transcription inhibitor's (IE2 fragment's) phosphorylation in vitro.

Administration of the IE2 fragment encoding DNA to a subject in need thereof can also be by gene-transfer technology. Such technologies include, but are not limited to, viruses, liposomes, and altered forms or derivatives of DNA or RNA.

V. Methods of Use

The present invention provides for the use of a transcription inhibitor in a subject to treat, limit or prevent an inflammatory disease or disorder or to treat, limit or prevent CMV infection or related disorders. Suitable transcription inhibitors are described above, as are suitable pharmaceutical compositions comprising said transcription inhibitors.

In one aspect, the present invention provides methods for inhibiting expression of a Spi-1 modulated molecule, e.g., proinflammatory cytokine, e.g., IL-1β or TNF in a cell comprising contacting the cell, e.g., a monocyte, with a sufficient amount of a transcription inhibitor.

In another aspect, the present invention provides a method of inhibiting inflammation by administering to a subject in need thereof a sufficient amount of a transcription inhibitor or pharmaceutical composition of the present invention.

In still another aspect, the present invention provides a method for treating an inflammatory disorder by administering to a subject in need thereof a therapeutically effective amount of an anti-inflammatory agent or pharmaceutical composition of the present invention. The inflammatory disorder contemplated by the present invention includes, but is not limited to, arthritis, rheumatoid arthritis, autoimmune diseases (e.g., inflammatory bowel disease and Systemic Lupus Erythematosus), solid organ transplantation, acute infection, acute phase response, allergic asthma, anorexia, asthma, cachexia, atherosclerosis, resolving myocardial infarction, coagulation, fever, gingivitis, graft versus host disease, hemorrhage, multiple sclerosis, neovascular glaucoma, osteoarthritis, periodontitis, psoriasis, psoriatic arthritis, rheumatic fever, shock, and solid tumor growth and tumor invasion by secondary metastases.

In yet another embodiment, the present invention provides a method for treating inflammatory disorders by administering to a subject in need thereof a therapeutically effective amount of a transcription inhibitor or pharmaceutical composition of the present invention in combination with one or more additional anti-inflammatory agents including, but not limited to, non-steroidal anti-inflammatory agents (e.g., NSAIDS), aspirin, corticosteroids, selective COX-2 inhibitors, interleukin-1 antagonists, dihydroorotate synthase inhibitors, p38 MAP kinase inhibitors, TNF-α inhibitors, TNF-α sequestration agents, and methotrexate.

The present invention also provides methods for treating a disease or disorder associated with CMV infection in a subject comprising administering to the subject a therapeutically effective amount of a transcription inhibitor or a pharmaceutical composition of the present invention. In one embodiment, the disease or disorder associated with CMV is selected from the group consisting of: CMV retinitis, hepatitis, CMV-associated acute transverse myelitis, mononeuropathy multiplex, encephalitis, fever, rash, and fatigue.

In another aspect, the present invention provides methods for inhibiting CMV expression or infection in a cell comprising contacting the cell with a polypeptide/IE2 fragment of the invention. In a related aspect, the present invention provides methods for treating, preventing, or limiting a CMV viral infection in a subject comprising administering to the subject a therapeutically effective amount of a polypeptide/IE2 fragment, an anti-inflammatory agent or a pharmaceutical composition of the present invention.

A therapeutically effective amount of a transcription inhibitor, for example as comprised in a pharmaceutical composition, may be administered by any known route of administration, including, but not limited to, aerosol (e.g., nasal or pulmonary), oral, subcutaneous, topical, intraocular, intramuscular, intravenous, intrathecal, etc.

The transcription inhibitor may be administered as a single dose, or in multiple doses administered over time. Non-limiting examples of intervals between multiple doses include up to 4 hours, up to 8 hours, up to 12 hours, up to 24 hours, up to 36 hours, up to 48 hours, up to 72 hours, up to one week, up to two weeks, up to one month, up to two months, up to three months, up to four months, up to six months, and up to one year.

An effective amount of a transcription inhibitor may be administered either prophylactically, or in the context of a person suffering from a disease or disorder, or to a person at risk for an inflammatory disease or disorder or known or suspected to have been exposed to CMV virus.

The present invention is further illustrated by the following non-limiting examples.

### EXAMPLES

Methods and Materials

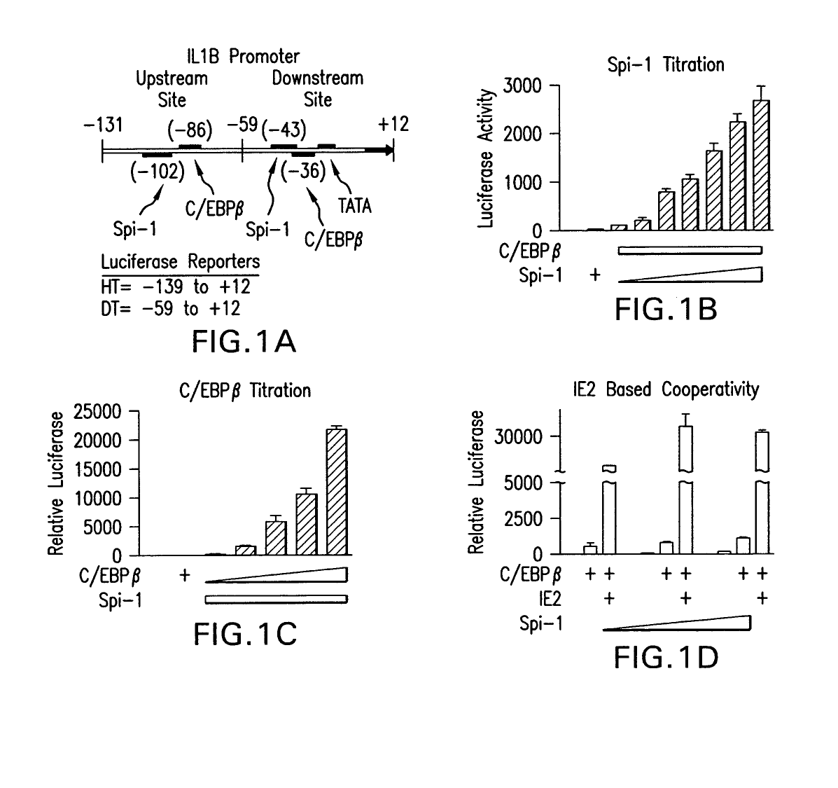

Cell Culture. HeLa cells (Strain S3) were obtained by ATCC and cultured per their recommendations. Briefly, the cells were grown in Dulbecco modified Eagle medium (DMEM) containing 10% FBS and 0.5% penicillin-streptomycin. Every three days cells were split 1:10 using Trypsin (0.25%) EDTA (0.1%) (Cellgro) to detach the cells.
Reporter Constructs and Expression Vectors. The human IL1B promoter regions (−131+12 and −59/+12 and its mutants (FIG. 1A) were generated by polymerase chain reaction (PCR) and inserted into pGL3-Basic vector (Promega) at Mlu I and BgI II or Hind III sites to construct promoter-luciferase reporter plasmids. The HCMV IE expression vectors pEQ273 and pEQ326 contain the genomic HCMV IE DNA inserted into pGEM1 vector. The plasmids expressing full-length NFIL6 and a truncated version with an internal deletion between the two Spl I restriction sites were constructed by inserting the NFIL6 cDNAs into expression vectors pcDNA3.1 or pcDNA1 (Invitrogen) (Tsukada, J., K. Saito, et al. (1994) Mol Cell Biol 14(11): 7285-97). The expression vector containing the bZIP region of NFIL6 was constructed by inserting a PCR-amplified fragment of NFIL6 encoding amino acids 269-345 into pcDNA3.1 (Yang, Z., N. Wara-Aswapati, et al. (2000) J Biol Chem 275(28): 21272-7).
Transfections and Reporter Gene Assays. HeLa (S3) cells were transfected with expression vectors and luciferase reporter plasmids using Effectene reagents (Qiagen Inc.). Cells were seeded into 24-well plates 24 hours before transfection. To ensure comparable transfection efficiency among wells, total amount of transfected DNA per well was kept constant within each experiment by adding various amounts of parental vectors and/or the vectors expressing either NFIL6 or IE proteins. Expression from these vectors was confirmed by western blotting. Twenty-four or forty-eight hours after transfection, the activities of luciferase and β-galactosidae activity were determined (using reagents from Promega) to measure promoter activity and allow normalization of luciferase activity in each experiment, respectively. Error bars represent the standard error for a minimum of three repetitions.
Purification of fusion proteins and GST Pull Down Assays. GST fusion proteins were harvested from E. coli BL21 (DE)pLysS (Promega, Madison, Wis.) using previously described methods (Wara-aswapati, 1999). Briefly, cultures were induced with 0.5 mM IPTG for 3-4 hours, pellets were suspended in NETN buffer (20 mM Tris, 100 mM NaCl, 1 mM EDTA, 0.05% NP-40) with lmM DTT, PefaBloc (Roche, Indianapolis, Ind.), and one Complete™ Protease Inhibitor Cocktail (Roche) tablet/50 ml. Suspensions were sonicated on ice and supernatants collected. Glutathione-Sepharose beads were washed in NETN buffer and incubated with fusion proteins at 4° C. rotating overnight. Beads were washed 3 times with NETN buffer and incubated with the in vitro translated protein probe of interest for 45 min rotating at 4° C. After 3 washes with ice cold NETN, beads were boiled and separated by SDS-PAGE. Gels were stained with SimplyBlue Safestain (Invitrogen) for protein determination and analyzed for binding using autoradiography. Radioloabeled protein probes were synthesized in vitro (TNT T7 Quick Coupled Reticulocyte Lysate System, Promega) and labeled with ³⁵S methionine (Amersham) according to the manufacturer's instructions.

### Example 1

### Inhibition of Spi-1 Function on IL-1β by IE2 Fragments

To test inhibition of Spi-1 function on the IL-1β promoter by IE2 291-364, a gene reporter assay was used. In this system the IL-1β promoter was spliced in front of a reporter gene, which encodes firefly luciferase. When the promoter is activated by Spi-1, the cell will produce the luciferase enzyme. The enzyme activity can then be measured by how brightly the cells glow. This common technology was employed because it is an easier and more sensitive method to measure promoter function than to measure gene products such as IL-1β.

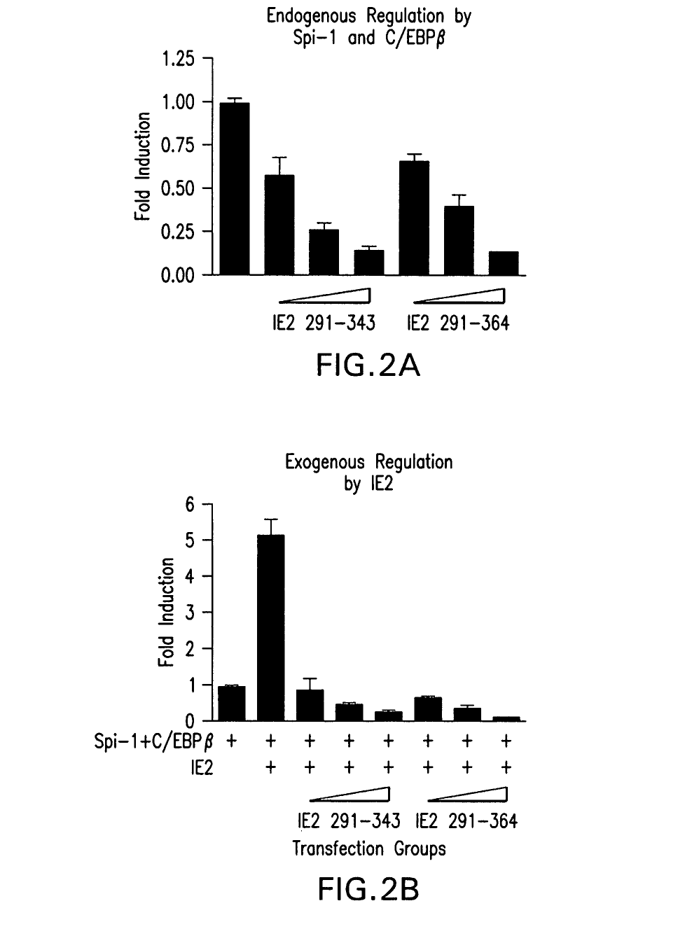

When Spi-1 and C/EBPβ were transfected into HeLa-S3 cells, the reporter showed a titratable response (FIGS. 1B & C). However, when full length wild-type IE2 was added to the cell transfection, the activity of the gene increased 4-10 fold (FIG. 1D). This demonstrated the potency of IE2. But if the inhibitor peptide was titrated in, there was diminishing activity of the reporter both in the absence and presence of full-length IE2 (FIGS. 2A-2B).

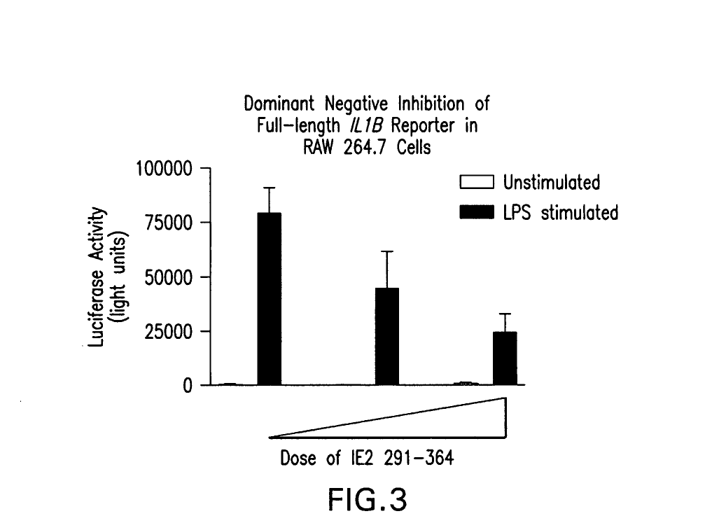

To further verify the effect of IE2 291-364, a test was conducted in the RAW 264.7 cell line. T his cell line has been demonstrated to respond to LPS induction of both endogenous and transfected IL1B genes (Shirakawa, F., K. Saito, et al., Id.) and expresses abundant levels of Spi-1 (Kominato, 1995) and C/EBPβ (Tsukada, 1994). When the RAW 264.7 cell line was treated with the bacterial cell wall product LPS, it produced IL-1β similar to what normal monocytes would do. The IE2-291-364 peptide inhibited IL-1β promoter reporter by about 50% in this system (FIG. 3).

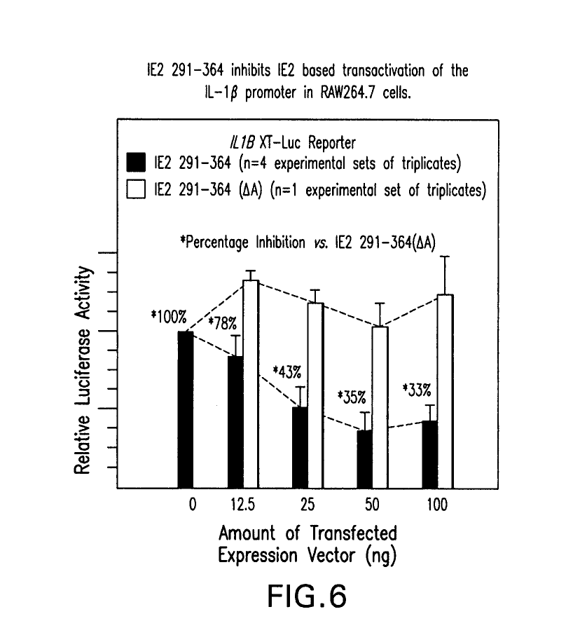

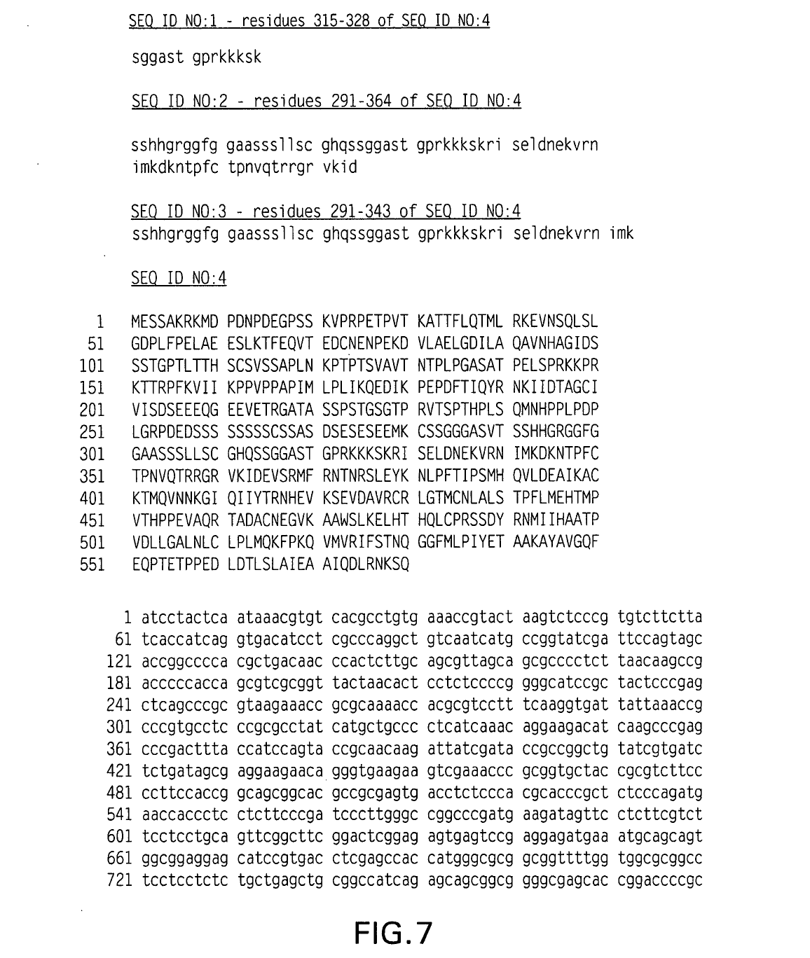

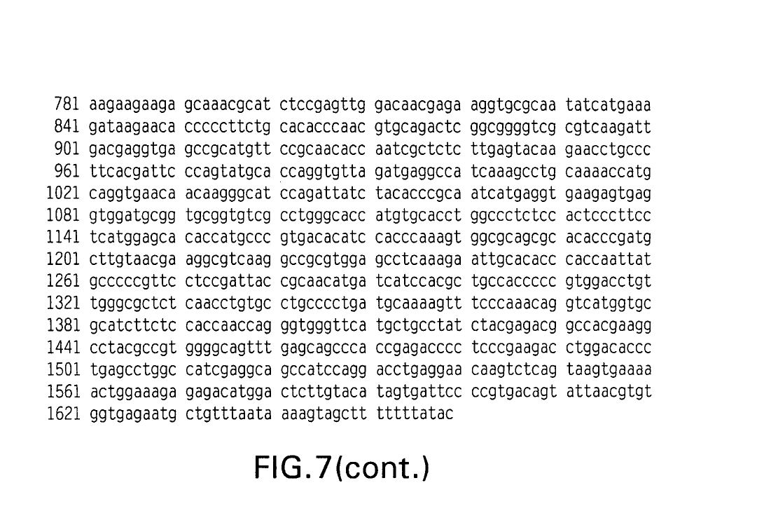

In order to more rigorously test the inhibitory capabilities of the IE2 peptides on IL1B gene expression, a complete monocyte-specific 3.8 kbp regulatory region of the IL1B gene containing both the LPS-responsive enhancer and the cell-type specific Spi-1-dependent promoter (Shirakawa, F., K. Saito, et al. (1993). Mol Cell Biol 13(3): 1332-44) was transfected as a luciferase reporter into LPS-treated RAW264.7 monocyte cells. The results show that cotransfection of the 3.8 kbp complete IL1B regulatory region with the IE2 291-364 peptide expression vector containing the His tag resulted in dose-dependent inhibition of activity. Maximum inhibition is between 50 and 75% relative to the reporter in the absence of the IE2 vector, and a similar degree of inhibition when compared to cotransfection with delA, a control vector containing the IE2 291-364 region and an internal deletion of residues 315-328 (FIG. 6).

### Example 2

### Expression of IE2 Fragments in the Transfection Assays

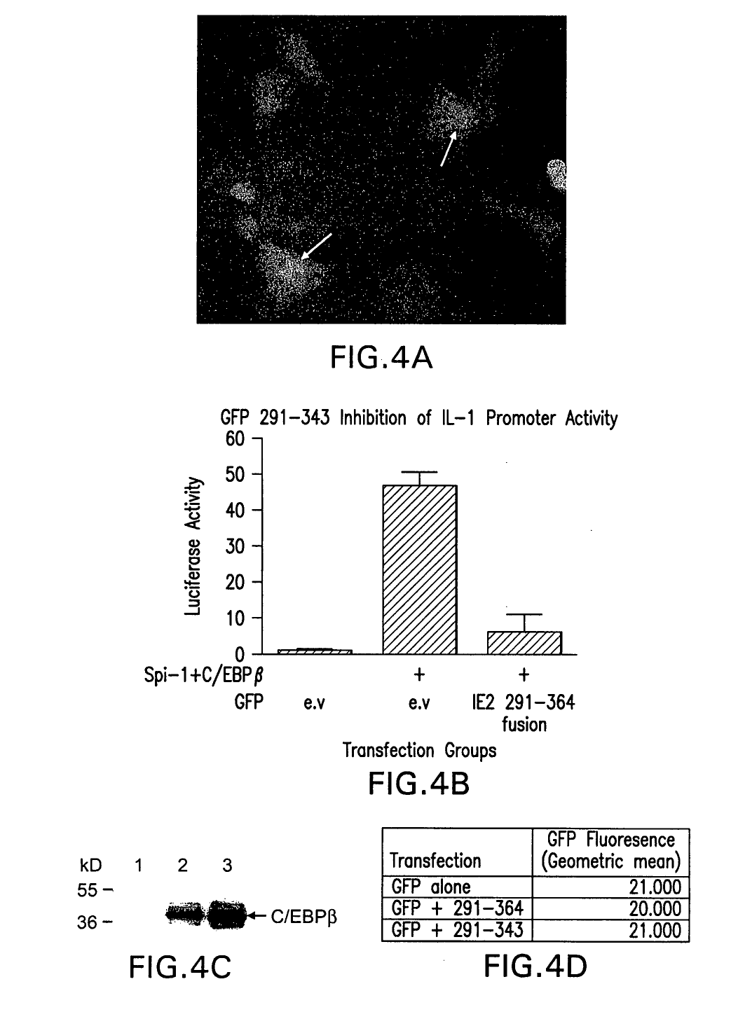

In order to confirm the expression of IE2 fragments in the transfection assays, the fragments were inserted into a GFP expression vector to determine if they would localize to the nucleus (FIG. 4A). This would indicate both expression of the protein as well as function of a putative nuclear localization sequence located within the IE2 fragments.

24 hours post transfection, the GFP 291-364 fusion product was found in both the cytoplasm and nucleus. The ability of the GFP 291-364 fragment to inhibit endogenous transactivation of the HT reporter by Spi-1 and C/EBPβ was then tested. To control for transfection efficiency, GFP expressing cells were first purified by FACS. Equal numbers of cells were then used to prepare lysates for the reporter assay. These data showed that both of the GPF fusion products retained a dominant negative function (FIG. 4B). This function was specific to the inserted IE2 fragments as there was generous reporter activity present when cotransfecting Spi-1 and C/EBPβ with the empty GFP expression vector (e.v.).

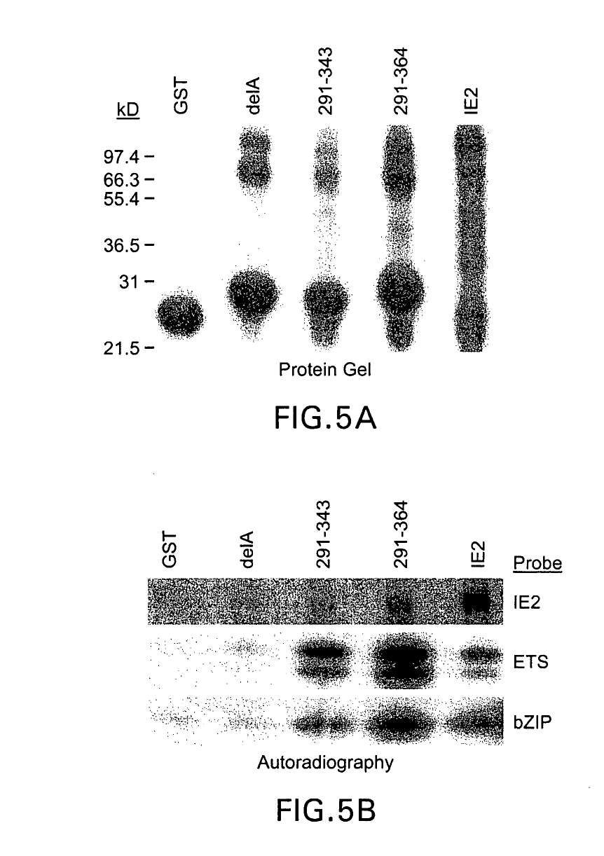

Because the IE2 291-364 region was previously mapped as one of three TBP binding sites on IE2, it was possible that the inhibitory function was due to interaction with TBP. To exclude this possibility, the expression of two other genes that contain TATA boxes in their promoters were investigated. First, an observed the expression of C/EBPβ from the expression vector by western blot (FIG. 4C) was conducted. This showed no reduction in C/EBPβ protein by co-expression of GFP 291-364. Second, His-tagged inhibitors were cotransfected with ev GFP and the geometric mean brightness of transfected cells measured by flow cytometry (FIG. 4D). Neither inhibitor reduced GFP expression, which illustrated that inhibition of IL-1β promoter activity was not due to a generalized interaction between the IE2 fragment inhibitors and TBP. Thus, the present Example demonstrated the capacity to express peptide fragments of the IE2 molecule that can inhibit the expression of the IL-1β gene by competitively interacting with Spi-1 and C/EBPβ. It is believed that the basis for the interaction is localized to residues 315-328 of the IE2 molecule because a variant of IE2 291-364 missing the 315-328 region (delA) lacks binding to Spi-1 ETS domain in a GST fusion pull-down assay (FIG. 5).

While the present disclosure is susceptible to various modifications and alternative forms, specific example embodiments have been shown in the Figures and are herein described in more detail. It should be understood, however, that the description of specific example embodiments is not intended to limit the invention to the particular forms disclosed, but on the contrary, this disclosure is to cover all modifications and equivalents as defined by the appended claims.

All patents, patent applications, publications, products descriptions, and protocols, and references cited herein are incorporated by reference for all purposes, and specifically for a referenced method or procedure.

What is claimed is:

1. An anti-inflammatory agent comprising an isolated polypeptide that comprises human Cytomegalovirus (hCMV) immediate early 2 (IE2) protein amino acid residues 315-328 (IE2 315-328) (SEQ ID NO:1), wherein said polypeptide inhibits inflammation.

2. A polypeptide selected from the group consisting of: human Cytomegalovirus (hCMV) immediate early 2 (IE2) protein amino acid residues 291-364 (IE2 291-364) (SEQ ID NO:2), hCMV IE2 protein amino acid residues 291-343 (IE2 291-343) (SEQ ID NO:3), and hCMV IE2 protein amino acid residues 315-328 (IE2 315-328) (SEQ ID NO:1).

3. A pharmaceutical composition comprising a therapeutically effective amount of a polypeptide according to claim 2 and a pharmaceutically acceptable carrier.

4. A polypeptide having an amino acid sequence at least 90% homologous to amino acid residues 291-364 (IE2 291-364) (SEQ ID NO:2).

5. A polypeptide having an amino acid sequence at least 90% homologous to amino acid residues (IE2 291-343) (SEQ ID NO:3).

6. A polypeptide having an amino acid sequence at least 90% homologous to amino acid residues IE2 315-328) (SEQ ID NO:1).

7. A fragment of an IE2 polypeptide between 14 and 75 amino acid residues in length which comprises the amino acid sequence of SEQ ID NO:1.

8. A pharmaceutical composition comprising a polypeptide according to claims 4, 5, 6, or 7, and a pharmaceutically acceptable carrier.

9. A pharmaceutical composition comprising a peptidomimetic capable of inhibiting transcription of an Spi-1-modulated molecule.

10. A method of inhibiting inflammation in a subject in need thereof, comprising administering to the subject a sufficient amount of a pharmaceutical composition comprising a polypeptide of claim 2, 4, 5, 6, or 7.

11. A method for treating an inflammatory disorder in a subject in need thereof, comprising administering to the subject a therapeutically effective amount of a pharmaceutical composition comprising a polypeptide of claim 2, 4, 5, 6, or 7.

12. The method of claim 11, wherein the inflammatory disorder is selected from the group consisting of arthritis, inflammatory bowel disease, Systemic Lupus Erythematosus, solid organ transplantation, acute infection, acute phase response, allergic asthma, anorexia, asthma, cachexia, atherosclerosis, resolving myocardial infarction, coagulation, fever, gingivitis, graft versus host disease, hemorrhage, multiple sclerosis, neovascular glaucoma, osteoarthritis, periodontitis, psoriasis, psoriatic arthritis, rheumatic fever, rheumatoid arthritis, shock, and solid tumor growth and tumor invasion by secondary metastases.

13. A method for treating an inflammatory disorder in a subject in need thereof, comprising administering to the subject a therapeutically effective amount of a pharmaceutical composition comprising a polypeptide of claim 2, 4, 5, 6, or 7 in combination with one or more additional anti-inflammatory agents.

14. The method of claim 13, wherein the additional anti-inflammatory agent is selected from the group consisting of: selective COX-2 inhibitors, corticosteroids, interleukin-1 antagonists, dihydroorotate synthase inhibitors, p38 MAP kinase inhibitors, TNF-α inhibitors, TNF-α sequestration agents, and methotrexate.

15. A method for treating a disease or disorder associated with Cytomegalovirus (CMV) infection in a subject comprising administering to the subject a therapeutically effective amount of a pharmaceutical composition comprising a polypeptide of claim 2, 4, 5, 6, or 7.

16. The method of claim 15, wherein the disease or disorder associated with CMV is selected from the group consisting of: Cytomegalovirus (CMV) retinitis, hepatitis, CMV-associated acute transverse myelitis, mononeuropathy multiplex, encephalitis, fever, rash, and fatigue.

17. A method for inhibiting Cytomegalovirus (CMV) expression in a cell comprising contacting the cell with the polypeptide of claim 2, 4, 5, 6, or 7.

18. A method for inhibiting expression of a proinflammatory cytokine in a cell comprising contacting the cell with the polypeptide of claim 2, 4, 5, 6, or 7.

19. The method of claim 17, wherein the cell is a monocyte.

20. The method of claim 17, wherein the proinflammatory cytokine is IL-1β.

21. The method of claim 17, wherein the proinflammatory cytokine is TNF.

22. A method for treating, preventing or limiting a Cytomegalovirus (CMV) viral infection in a subject comprising administering to the subject a therapeutically effective amount of a pharmaceutical composition comprising a polypeptide of claim 2, 4, 5, 6, or 7.


SEQUENCE LISTING (6 sequences)

SEQ ID NO: 1 - protein, 14 aa, artificial sequence
  synthetic peptide
SGGASTGPRKKKSK

SEQ ID NO: 2 - protein, 74 aa, artificial sequence
  synthetic peptide
SSHHGRGGFGGAASSSLLSCGHQSSGGASTGPRKKKSKRISELDNEKVRNIMKDKNTPFCTPNVQTRRGRVKID

SEQ ID NO: 3 - protein, 53 aa, artificial sequence
  synthetic peptide
SSHHGRGGFGGAASSSLLSCGHQSSGGASTGPRKKKSKRISELDNEKVRNIMK

SEQ ID NO: 4 - protein, 580 aa, Human herpesvirus 5
MESSAKRKMDPDNPDEGPSSKVPRPETPVTKATTFLQTMLRKEVNSQLSLGDPLFPELAEESLKTFEQVTEDCNENPEKDVLAELGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 5 - DNA, 1659 bp, Human herpesvirus 5
atcctactcaataaacgtgtcacgcctgtgaaaccgtactaagtctcccgtgtcttcttatcaccatcaggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcacctggccctctccactcccttcctcatggagcacaccatgcccgtgacacatccacccaaagtggcgcagcgcacacccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccacgaaggcctacgccgtggggcagtttgagcagcccaccgagacccctcccgaagacctggacaccctgagcctggccatcgaggcagccatccaggacctgaggaacaagtctcagtaagtgaaaaactggaaagagagacatggactcttgtacatagtgattccccgtgacagtattaacgtgtggtgagaatgctgtttaataaaagtagctttttttatac

SEQ ID NO: 6 - protein, 11 aa, artificial sequence
  synthetic peptide
YARAAAAQARA